(19) United States
(12) Patent Application Publication
(10) Pub. No.: US 2006/0149363 A1
(43) Pub. Date: Jul. 6, 2006

(54) Optimally expanded, collagen sealed ePTFE graft with improved tissue ingrowth

(75) Inventor: Dennis Kujawski, Warwick, NY (US)

Correspondence Address: HOFFMANN & BARON, LLP, 6900 JERICHO TURNPIKE, SYOSSET, NY, 11791, US

(73) Assignee: SCIMED Life Systems, Inc.

(21) Appl. No.: 11/030,346

(22) Filed: Jan. 6, 2005

(51) Int. Cl. A61F 2/06; A61F 2/02

(52) U.S. Cl. 623/1.38; 623/1.39; 623/23.75

(57) ABSTRACT

The present invention provides An implantable ePTFE artificial vascular graft comprising a tubular structure comprising a highly expanded ePTFE material having a porous structure, and a sealant saturated within said porous structure of said ePTFE material to make it substantially non-porous.

### FIELD OF INVENTION

The present invention relates generally to a tubular implantable prosthesis such as vascular grafts and endoprostheses formed of porous polytetrafluoroethylene. More particularly, the present invention relates to a highly expanded PTFE graft including a reabsorbable sealing material for providing internodal sealing during the intraoperative and immediate postoperative time periods while supporting transmural tissue growth by the degradation over time of the reabsorable sealant material.

### BACKGROUND OF THE INVENTION

Implantable prostheses are commonly used in medical applications. One of the more common prosthetic structures include tubular prostheses which may be used as vascular grafts to replace or repair damaged or diseased blood vessels. To maximize the effectiveness of such a prosthesis, it should be designed with characteristics which closely resemble that of the natural body lumen which it is repairing or replacing.

It is well known to use extruded tubes of polytetrafluoroethylene (PTFE) in such applications particularly as vascular grafts. PTFE is particularly suitable as an implantable prosthesis as it exhibits superior biocompatability. PTFE tubes may be used as vascular grafts in the replacement or repair of a blood vessel as PTFE exhibits low thrombogenicity. In vascular applications, the grafts are manufactured from expanded polytetrafluoroethylene (ePTFE) tubes. These tubes have a microporous structure which allows natural tissue ingrowth and cell endothelization once implanted in the vascular system. This contributes to long term healing and patency of the graft.

Grafts formed of ePTFE have a fibrous state which is defined by interspaced nodes interconnected by elongated fibrils. The spaces between the node surfaces that is spanned by the fibrils is defined as the internodal distance (IND). A graft having a large IND enhances tissue ingrowth and cell endothelization as the graft is inherently more porous.

It is also known in order to achieve in-growth, to use a porous material for tubular vascular grafts such as a textile material. While textile structures have the advantage of being naturally porous they do not possess the natural biocompatibility of ePTFE grafts.

The art is replete with examples of microporous ePTFE tubes useful as vascular grafts. While a significant advantage of ePTFE is its quality of fluid-tightness, certain advantages can be gained by providing for controlled blood flow through the prosthesis after initial implantation. Controlled blood flow through the walls of a prosthesis after implantation can support transmural tissue growth and angiogenesis. This ingrowth can provide a viable intima and possibly a graft with patency rates due to a consistent tissue to blood interface. Providing transmural blood flow and therefore transmural tissue growth can be achieved with an ePTFE graft by highly expanding the PTFE. The porosity of an ePTFE vascular graft can be controlled by controlling the IND of the microporous structure of the tube. An increase in the IND within a given structure results in enhanced tissue ingrowth as well as cell endothelization along the inner surface thereof. However, such increase in the porosity of the tubular structure also results in excessive blood loss during intra-operative period and can allow bleeding through the graft or seroma formation post-operatively.

One way in which the porosity of a graft can be controlled is to apply a natural coating, such as collagen or gelatin. It is desirable that a vascular graft ultimately be sufficiently blood-tight to prevent the loss of blood during implantation, yet also be sufficiently porous to permit in-growth of fibroblast and smooth muscle cells in order to attach the graft to the host tissue and ensure a successful implantation and adaptation within the host body.

It is therefore desirable to provide an ePTFE graft of highly expanded PTFE for supporting transmural tissue growth.

It is therefore further desirable to provide an ePTFE graft of highly expanded PTFE for supporting angiogenesis.

It is therefore further desirable to provide an ePTFE graft of highly expanded PTFE also comprising a resorbable sealant for providing a hemostatic ePTFE graft during implantation and the immediate postoperative time frame.

It is therefore further desirable to provide an ePTFE graft of highly expanded PTFE also comprising a sealant of collagen, gelatin or other biologically based degradable materials.

It is therefore further desirable to provide an ePTFE graft of highly expanded PTFE also comprising a sealant of non-biologic, degradeable material.

It is therefore further desirable to provide an ePTFE tubular vascular graft having a highly expanded layer whose porosity is sufficient to promote enhanced transmural cell growth and tissue incorporation, hence better patency rates due to a more consistent tissue to blood interface while providing a seal structure to prevent leakage during the implantation of the graft.

### SUMMARY OF THE INVENTION

It is an advantage of the present invention to provide an ePTFE graft of highly expanded PTFE for supporting transmural tissue growth.

It is an advantage of the present invention to provide an ePTFE graft of highly expanded PTFE for supporting angiogenesis.

It is an advantage of the present invention to provide an ePTFE graft of highly expanded PTFE also comprising a resorbable sealant for providing a hemostatic ePTFE graft during implantation and the immediate postoperative time frame.

It is an advantage of the present invention to provide an ePTFE graft of highly expanded PTFE also comprising a sealant of collagen, gelatin or other biologically based degradable materials.

It is an advantage of the present invention to provide an ePTFE graft of highly expanded PTFE also comprising a sealant of non-biologic, degradeable material.

It is an additional advantage of the present invention to provide an ePTFE tubular vascular graft having a highly expanded layer whose porosity is sufficient to promote enhanced transmural cell growth and tissue incorporation, hence better patency rates due to a more consistent tissue to blood interface while providing a seal structure to prevent leakage during the implantation of the graft.

In the efficient attainment of these and other advantages, the present invention provides An implantable ePTFE artificial vascular graft comprising a tubular structure comprising a highly expanded ePTFE material having a porous structure, and a sealant saturated within said porous structure of said ePTFE material to make it substantially non-porous.

In another embodiment, the present invention provides a method of making a temporarily blood-tight implantable ePTFE material for improved tissue ingrowth comprising providing an ePTFE material having an average internodal distance of about 60-100 micrometers; and saturating the ePTFE material with a biodegradable sealant.

The highly expanded ePTFE graft preferably may be used as a vascular graft. As more particularly described by way of the preferred embodiment herein, an ePTFE tubular structures is formed of highly expanded polytetrafluoroethylene (ePTFE). Further, the ePTFE tubular structure incorporates a sealant component. The sealant component may be incorporated into the structure of the graft, or may be layered on the interior or exterior walls of the structure. The amount and degree of sealant can be varied as required in accordance with alternate uses of the graft.

The sealing component may alternately be incorporated into the microstructure of the vascular graft or layered onto the interior or exterior of the graft material. Examples of such sealants include; collagen, gelatin, other biological based materials, or non-biological sealants could also be used.

### BRIEF DESCRIPTION OF THE DRAWINGS

FIG. 1 is a schematic representation of the microstructure of ePTFE material.

FIG. 2 and is a schematic representations of the microstructure of highly expanded ePTFE material.

FIG. 3 is a schematic representation of the microstructure of the sealed ePTFE material of the present invention.

### DETAILED DESCRIPTION OF THE PREFERRED EMBODIMENTS

While this invention may be satisfied by embodiments in many different forms, there will be described herein in detail, preferred embodiments of the invention, with the understanding that the present disclosure is to be considered as exemplary of the principles of the invention and is not intended to limit the invention to the embodiments illustrated and described.

The prosthesis of the preferred embodiments of the present invention is a tubular structure which is particularly suited for use as a vascular graft. The prosthesis is formed of extruded polytetrafluoroethylene (PTFE) as PTFE exhibits superior biocompatability.

PTFE is particularly suitable for vascular applications as it exhibits low thrombogenicity. Tubes formed of extruded PTFE may be expanded to form ePTFE tubes where the ePTFE tubes have a fibrous state which is defined by elongate fibrils interconnected by spaced apart nodes. Such tubes are said to have a microporous structure, the porosity of which is determined by the distance between the surfaces of the nodes, referred to as the internodal distance (IND). Tubes having a large IND (greater than 40 microns) generally exhibit better long term patency as the larger pores promote cell growth (cells may not be endothelium) along the inner blood contacting surface. Tubes having lower IND (less than 40 microns) exhibit inferior healing characteristics, however they offer superior radial tensile and suture retention strengths desirable in a vascular graft. The present invention provides a tubular structure which promotes long term patency of the graft by providing for enhanced cell proliferation and angiogenesis along the inner surface while exhibiting enhanced strength due to having a large IND, and a sealant included in the graft The graft morphology is consistent throughout the wall. Strength is provided by the highly expanded ePTFE structure alone. The sealant prevents intraoperative and post-operative bleeding).

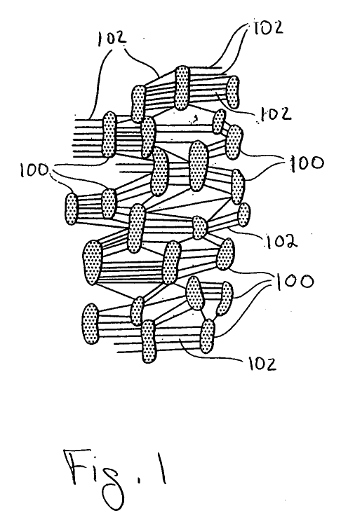

Porous ePTFE is well known in the art and is described in detail, for example, in U.S. Pat. Nos. 3,953,566 and 3,962,153, which is incorporated herein by reference as shown in FIG. 1. Generally, paste-forming techniques are used to convert the polymer in paste form to a shaped article which is then expanded, after removing the lubricant, by stretching it in one or more directions; and while it is held in its stretched condition it is heated to at least 348° C. after which it is cooled. The porosity that is produced by the expansion is retained for there is little or no coalescence or shrinking upon releasing the cooled, final article.

Paste-forming of dispersion polymerized poly(tetrafluoroethylene) is well known commercially. Extrusions of various cross-sectional shapes such as tubes, rods and tapes are commonly obtained from a variety of tetrafluoroethylene resins, and other paste-forming operations such as calendering and molding are practiced commercially. The steps in paste-forming processes include mixing the resin with a lubricant such as odorless mineral spirits and carrying out forming steps in which the resin is subjected to shear, thus making the shaped articles cohesive. The lubricant is removed from the extruded shape usually by drying.

The paste-formed, dried, unsintered shapes are expanded by stretching them in one or more directions under certain conditions so that they become substantially much more porous and stronger. Expansion and sintering increases the strength of PTFE resin within preferred ranges of rate of stretching and preferred ranges of temperature. It has been found that techniques for increasing the crystallinity, such as annealing at high temperatures just below the melt point, improve the performance of the resin in the expansion process.

The porous microstructure of the ePTFE material is affected by the temperature and the rate at which it is expanded. The structure consists of nodes 100 interconnected by very small fibrils 102. In the case of uniaxial expansion the nodes 100 are elongated, the longer axis of a mode being oriented perpendicular to the direction of expansion. The fibrils 102 which interconnected the nodes 100 are oriented parallel to the direction of expansion. These fibrils 102 appear to be characteristically wide and thin in cross-section, the maximum width being equal to about 0.1 micron (1000 angstroms) which is the diameter of the crystalline particles. The minimum width may be one or two molecular diameters or in the range of 5 or 10 angstroms. The nodes 100 may vary in size from about 400 microns to less than a micron, depending on the conditions used in the expansion. Products which have expanded at high temperatures and high rates have a more homogeneous structure, i.e., they have smaller, more closely spaced nodes 102 and these nodes 100 are interconnected with a greater number of fibrils 102.

When the ePTFE material is heated to above the lowest crystalline melting point of the poly(tetrafluoroethylene), disorder begins to occur in the geometric order of the crystallites and the crystallinity decreases, with concomitant increase in the amorphous content of the polymer, typically to 10% or more. These amorphous regions within the crystalline structure appear to greatly inhibit slippage along the crystalline axis of the crystallite and appear to lock fibrils and crystallites so that they resist slippage under stress. Therefore, the heat treatment may be considered an amorphous locking process. The important aspect of amorphous locking is that there be an increase in amorphous content, regardless of the crystallinity of the starting resins. Whatever the explanation, the heat treatment above 348° C. causes a surprising increase in strength, often doubling that of the unheated-treated material.

The preferred thickness of ePTFE material ranges from 0.025 millimeter to 2.0 millimeters; the preferred internodal distance within such ePTFE material ranges from 15 micrometers to 30 micrometers. The longitudinal tensile strength of such ePTFE material is preferably equal to or greater than 1,500 psi, and the radial tensile strength of such ePTFE material is preferably equal to or greater than 40 psi.

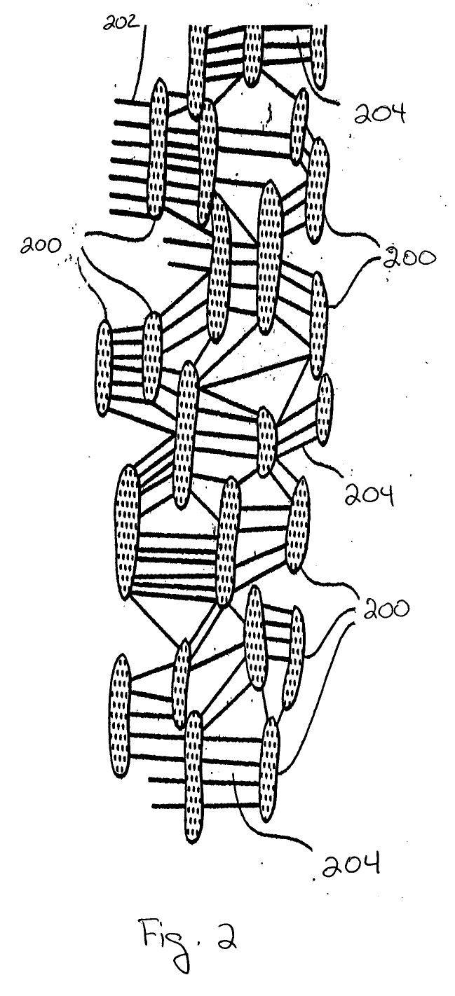

Turning now to FIG. 2 there is shown a highly expanded graft according to the present invention. Typically, an ePTFE tubular graft has an internodal distance in the range of 15 to 30 microns in order to provide a blood-tight graft. This is especially important during implantation as well as the immediate post operative time frame. However, it is advantageous to have internodal distances in the range of 30-100 microns in order to support transmural tissue growth and angiogenesis. Highly expanded ePTFE having internodal distances in the range of 30-100 microns according to the present invention provides enhanced ingrowth of a viable neointima by allowing for the passage of blood cells through the walls of the graft. The internodal distance range according to the present invention provide a consistent tissue to blood interface through the walls of the graft in order to support a viable intima, thus leading to higher patency rates. Turning to FIG. 2 there is shown a schematic representations of the microstructure of highly expanded ePTFE material according to the present invention. The structure consists of nodes 200 interconnected by very small fibrils 202. In the case of uniaxial expansion the nodes 200 are elongated, the longer axis of a mode being oriented perpendicular to the direction of expansion. The fibrils 202 which interconnected the nodes 200 are oriented parallel to the direction of expansion. In the highly expanded PTFE according to the present invention, the internodal distance, 204 is within the range of 30-100 microns. While the use of ePTFE grafts having IND's of greater than about 30 microns can promote the formation of a viable neointima and thus greater patency, the larger IND's allow excessive blood loss through the graft walls until the formation of the neointima. Therefore in order to utilize a graft with such an IND it is necessary to seal the graft in such a way that the sealant provides a hemostatic shield during implantation and during the immediate post operative time period. The sealant will then begin to degrade at a rate corresponding to the formation of the neointima. The degradation of the sealant provides for transmural tissue growth and thereby the formation of the neointima.

The highly expanded PTFE of the present invention is sufficiently porous to allow substantial ingrowth. The natural drawback however is that the porous structure is not initially blood-tight and hemorrhaging occurs. The present invention addresses that problem by providing a biocompatible mixture which makes the highly expanded material substantially non-porous and blood-tight, until such time as a neointima seals the graft. Typically, this time frame is approximately 6-8 weeks.

According to the present invention, the sealant material may be a biocompatible liquid which is easily saturated or impregnated within the fibrous region of the PTFE material. Such sealants include collagen and other biomaterials such as polyglycolic acid and polyactic acid.

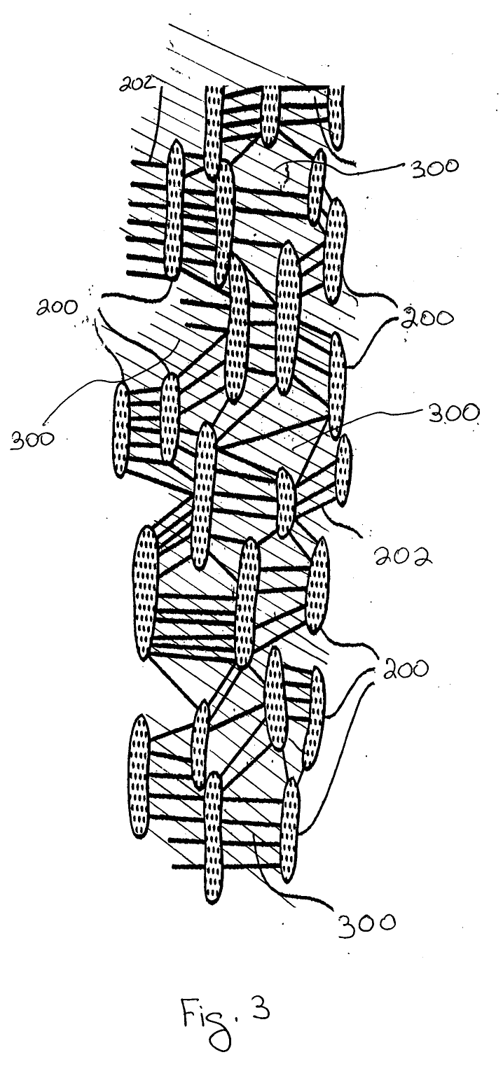

With reference now to the figures, FIG. 3 shows a photomicrograph of a highly expanded PTFE vascular graft. Mircopores 300 is saturated or impregnated with a sealant mixture in order to provide a blood-tight material, prosthesis, or more particularly a blood-tight artificial vascular graft.

The blood tight properties of the highly expanded PTFE material of the present invention may be used in other applications besides vascular grafts. The blood tight implantable highly expanded material may be used in many applications where it is desirable that an initially blood tight material may be needed; but also a material which further allows assimilation in the host body as the biodegradable impregnated material biodegrades in the body. A preferred embodiment is as a vascular patch. A vascular patch may be constructed of a thin layer membrane of the implantable highly expanded PTFE material which is generally in an elongate planar shape. As is well know, a vascular patch may be used to seal an incision in the vascular wall or otherwise repair a soft tissue area in the body.

Preferably, the biocompatible sealant is also bioresorbable. The implantable highly expanded PTFE material preferably at first is blood tight but over time the sealant degrades within the body and is reabsorbed by the body as highly expanded PTFE material is assimilated, i.e. in growth within the porous structure, incorporating it into the host body. Typically, highly expanded ePTFE would have permeabilities in excess of 500 cc/min/square cm., while sealed ePTFe such as in accordance with the present invention would have permeabilities between 0 and 5 cc/min/square cm.

In practicing the preferred method, the ePTFE starting material is initially in the form of a cylindrical tube. The length may vary depending on the intended end use.

Various methods can be described with respect to the application of the sealant, such as dip coating, spraying, or brushing. With respect to the present invention, the sealing process may be carried out by injecting collagen into the lumen and force the collagen through the wall by plugging up the distal end. The tube would be rolled to evenly disperse the collagen then the structure would be dried in an oven.

Various changes to the foregoing described and shown structures would now be evident to those skilled in the art. Accordingly, the particularly disclosed scope of the invention is set forth in the following claims.

What is claimed is:

1. A method of making a temporarily blood-tight implantable ePTFE material for improved tissue ingrowth comprising:
providing an ePTFE material having an average internodal distance of 60-100 microns; and
saturating said ePTFE material with a biodegradable sealant.

2. A method according to claim 1 wherein said ePTFE material is an artificial vascular graft.

3. A method according to claim 1 wherein said sealant is bioresorbable within the body after implantation.

4. A method according to claim 1 wherein said blood-tight implantable ePTFE material has a porosity of impregnation of less than about 5 cc/min/square cm.

5. A method according to claim 1 wherein said mixture is saturated within said ePTFE material by massaging said mixture into pores of said ePTFE material.

6. A blood-tight ePTFE material implantable in a mammal comprising:
an highly expanded ePTFE material having a porous structure and, a sealant saturated within said porous structure of said ePTFE material to make it substantially non-porous.

7. A blood-tight ePTFE material according to claim 6 wherein said sealant is collagen.

8. A ePTFE material according to claim 6 wherein said blood-tight ePTFE material is a vascular graft.

9. A blood-tight ePTFE material according to claim 6 wherein said sealant is bioresorbable within the body after implantation.

10. A blood-tight ePTFE material according to claim 6 wherein said blood-tight implantable ePTFE material has a porosity of impregnation of less than about 5 cc/min/square cm.

11. An artificial vascular graft comprising:
a tubular structure comprising a highly expanded ePTFE material having a porous structure, and a sealant saturated within said porous structure of said ePTFE material to make it substantially non-porous.

12. An artificial vascular graft according to claim 11 wherein said sealant is collagen.

13. An artificial vascular graft according to claim 11 wherein said blood-tight ePTFE material is a vascular graft.

14. An artificial vascular graft according to claim 11 wherein said sealant is bioresorbable within the body after implantation.

15. An artificial vascular graft according to claim 11 wherein said blood-tight implantable ePTFE material has a porosity of impregnation of less than about 5 cc/min/square cm.
